(19) United States
(12) Patent Application Publication
(10) Pub. No.: US 2002/0187519 A1
(43) Pub. Date: Dec. 12, 2002

(54) Treatment regimen for prostate cancer

(76) Inventor: Larry Helson, Quakertown, PA (US)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/208,934

(22) Filed: Jul. 30, 2002

Related U.S. Application Data
(62) Division of application No. 09/512,435, filed on Feb. 25, 2000.
(60) Provisional application No. 60/121,920, filed on Feb. 26, 1999.

(51) Int. Cl. G01N 33/574; A61K 31/56; A61K 31/337

(52) U.S. Cl. 435/7.23; 514/449; 514/171

(57) ABSTRACT

This invention provides for a novel schedule and dosage regimen of the combination paclitaxel-estramustine and chemical or surgical androgen depletion to treat patients with advanced prostate cancer who have not received previous therapy and are candidates for hormonal therapy. This invention differs from all current treatment strategies by the intercalation of cytotoxic chemotherapy (preferably paclitaxel and estramustine) at a specified interval (two months; range 1-6 months) after initiation of androgen depletion therapy.

### CROSS-REFERENCE TO A RELATED APPLICATION

This application is a divisional of co-pending U.S. Ser. No. 09/512,435, filed Feb. 25, 2000 which claims priority to provisional application Serial No.60/121,920 filed Feb. 26, 1999.

### FIELD OF THE INVENTION

The present invention broadly concerns a treatment regimen in patients with prostate cancer. More specifically, the present invention relates to a therapeutic program or treatment schedule to juxtapose two different anticancer modalities; hormonal or surgical depletion of endogenous androgens and a combination of cytotoxic drugs, preferably including a taxane, to patients with prostate cancer in an effort to stabilize, reduce, or destroy the cancerous growth. Specifically, the present invention concerns the timing of the introduction of cytotoxic chemotherapy, most preferably a combination of paclitaxel and estramustine, during hormonal manipulation.

### BACKGROUND OF THE INVENTION

Prostate cancer is the second most frequently occurring cancer in the male population in the United States after skin cancer. The 185,000 new cases diagnosed in 1988 represented 25% of all newly diagnosed cancers. During this same year approximately 40,000 men died because of the disease, and it remains the second leading cause of cancer-related death in men.

The basic strategy of initial therapy for this disease ranges from watchful waiting to surgical or chemical castration. Androgen deprivation has been the mainstay of prostate cancer treatment and is often the only therapy offered to patients. The current standard of care for Stage II or higher risk disease includes some form of androgen depletion based on the assumed androgen dependency of prostatic tumors in the majority of men at the time of diagnosis. This approach has not changed in over 30 years. The majority of patients respond to this treatment temporarily, and virtually all relapse by two years. Inevitably, tumor control is lost, the tumor burden becomes even larger than it was at the time of diagnosis, and in addition it is characterized by an androgen-independent phenotype. Following relapse, patients are offered palliative radiation, steroids, and cytotoxic chemotherapies. Application of these treatment options in this sequential manner, and in particular new chemical entities given alone or in combination, may have some impact on patient quality of life. However, responses in these circumstances are brief, limited in extent, and have little impact on survival. This serial application of different therapeutic modalities in response to increasing tumor burdens, a fundamental characteristic of prior art strategy, is flawed. It is essentially a conservative clinical status-driven response strategy. Both prophylaxis and a risk-driven strategy for this disease do not exist.

Peereboom et al. (1996) reported the results of phase I trials of patients having hormone-refractory prostate cancer (“HRPC”; androgen ablatement had failed) who were administered estramustine (EMP) at 600 mg/m²/d and escalating doses of paclitaxel (70, 135, 175, 210 mg/m²) by 3-hour infusion every three weeks. Of 15 evaluable patients, 3 had PSA declines over 50%, 7 had declines of 33-48%, 2 had stable disease, and 3 had progression.

Smith et al. (1997) reported the results of a phase II trial of HRPC patients who presented disease progression following primary androgen therapy and withdrawal from anti-androgens. Patients were administered estramustine 280 mg PO tid and etoposide 50 mg PO bid on days 1-14 with paclitaxel 135 mg/m² IV over 1 hour on day 2 of a 21-day cycle. Cycles were repeated to a maximum of 6. Of 23 patients evaluable for response, 12 had a decline in PSA of 50% or more.

Hudes et al. (1997) reported on a phase II trial of 96-hour paclitaxel infusion at 120 mg/m² over days 1-4 of a 21-day cycle, together with daily oral administration of estramustine at 600 mg/m²//d, continuously. All patients had hormone-refractory prostate cancer. Although 17 of 32 patients in the study had a. 50% decline of PSA, median time to progression, based on increasing PSA level and other clinical criteria, was only 22.5 weeks.

Garay et al. (1998) reported the results of a phase I trial wherein patients having HRPC received a fixed daily dose of EMP at 600 mg/m² for 6 weeks and paclitaxel as a 3-hour intravenous infusion (with dexamethasone premedication) weekly for 6 weeks, q 8 weeks. The dose of paclitaxel was escalated from 60 to 118 mg/m²/week. Five of eight patients had 75% decrease from baseline PSA.

The predominance of androgen-dependent tumors at diagnosis coupled with the emergence of androgen-independent tumors after apparently successful initial therapy can be explained as follows. With unrelenting tumor growth, genetic instability and mutations accumulate sufficiently to allow androgen-independent growth mechanisms to occur de novo, or to be expressed phenotypically in the parental lines, and in later generations of tumor cells. This transformation of tumor cell populations presumably occurs immediately after hormonal therapy is introduced. Based on clinical relapses within the first two years of hormonal therapy, the rate and accumulation of such cells is a continuous process and is characterized by a Gompertzian type of growth curve. There is thus a continuing need for new treatment regimens for prostate cancer.

The subject invention concerns a novel treatment regimen for patients diagnosed with prostate cancer whereby chemotherapy is administered in conduction with hormonal therapy. In practice, under standard androgen ablatement regimens a patient□s general demeanor is vastly improved about the eighth week after hormonal therapy is initiated. This coincides with maximum depletion of androgen-dependent tumor (minimal tumor burden). According to the subject invention, this is the preferred time to introduce cytotoxic chemotherapy. Superimposing cytotoxic chemotherapy during a hormonal induction is contrary to prior art standards of care, and has no precedent in the treatment of prostate cancer. The clinical significance of this invention is based upon the current incidence, prevalence, and outcomes of prostate cancer patients in the United States.

### SUMMARY OF THE INVENTION

It is an object of the present invention to provide a new and useful method for administration of cytotoxic chemotherapy and hormonal therapy to prostate cancer patients. It is a further object of the present invention to increase the duration of clinically significant responses in prostate cancer patients. Still a further object of the present invention is to provide a method allowing optimum cytotoxic activity against prostate cancer. These and other objects of the present invention are accomplished by new methods of administration of cytotoxins, preferably, taxanes and estramustine.

This invention discloses a method for treating prostate cancer with a combined application of any active hormonal therapy and any efficient cytotoxic chemotherapy at prescribed intervals. The time of administration of the effective drug combination at minimal tumor burden, usually at about the eighth week of androgen ablative therapy, is the critical feature of this invention. The novel combination of estramustine administration coupled with a recently patented scheduling for paclitaxel (Ainsworth et al., U.S. Pat. No. 5,696,153) is also a part of the most preferred embodiment of this invention, but can be modified or replaced in the event that more efficient cytotoxic therapies are developed. In a preferred embodiment, paclitaxel is administered to the patient within a range of 45-135 mg/m² over a duration of 60-180 minutes for a plurality of times during a 21-day period, with each of these administrations being separated from the next by an interval of approximately 4-5 days. Preferably, this protocol is repeated for at least three (3) cycles of 21 days (63 days total) with the plurality of times of infusing the paclitaxel beginning on the second day of each of the cycles. The protocol can be repeated for more than 3 cycles, for example, 6 cycles or more, so long as the patient can tolerate the toxic effects. Preferably, the duration of each infusion is between about 60-80 minutes and there are three (3) treatment days in each cycle separated by four (4) non-treatment days. Moreover, it is preferred that the amount of paclitaxel infused be about 60 mg/m².

These treatments do not engender serious adverse drug-related effects because of the improved therapeutic efficiency and the absence of a need for excessive dosing to the maximally tolerated level.

### DETAILED DESCRIPTION OF THE PREFERRED EMBODIMENT

The subject invention is a result of the discovery that a fundamental shortcoming of the prior art treatments for prostate cancer lies in the pattern of treatment scheduling, rather than in the inadequacy of available treatment modalities. In this invention, I have designed a novel pattern of therapy taking into account the two most critical factors, selection of and timing of introduction of cytotoxic chemotherapy. The rationale and the utility of two preferred types of drugs, taxanes (preferably paclitaxel) and estramustine, has been amply described (1-4, 14, 15). While estramustine is essentially palliative and has limited impact on the course of androgen-independent prostate cancer (“AIPC”, also can be termed “hormone-refractory prostate cancer” or “HRPC”), its immediate target is microtubule associated proteins making it a mitotic inhibitor (5-8). These observations have suggested that it might have complementary activity in combination with other mitotic inhibitors like paclitaxel. Preclinical (9) and pilot clinical trials (10) support this hypothesis. While no evidence of prolongation of survival has of yet been demonstrated, subjective responses as high as 60% have been reported (4) and attest to the fact that these two drugs combined may represent the best available synergistic therapeutic combination for prostate cancer. In view of the current state of the art□s ability to administer any variety of combinations of taxanes and estramustine, this invention is the concept that scheduling of administration is critical in differentiating the combination from a palliative application at failure of hormonal therapy, to a novel prophylactic treatment modality prior to failure as taught herein. In most cases, after induction with androgen ablative agents or procedures, about the eighth week of therapy coincides with maximum decrease in tumor burden (but this can vary from 2 weeks to 6 months after induction). According to the teachings herein, it is at this time that cytotoxic chemotherapy, preferably with a combination of paclitaxel and EMP, should begin. There follows diminished risk of normal tissue toxicity from the drug combination introduced at this time as well. This means that patients will have responded well enough at this time to tolerate a moderate chemotherapeutic treatment better than when they have relapsed and are in less advantageously positioned clinical states. There is also maximal efficiency in tumor cytotoxic effects because of the minimal tumor burden and increased residual cell cycling activity at this time in the majority of patients. There is the added benefit that androgen-independent tumor cells have enhanced cycling activity, and better vascularity and oxygenation, all of which improve the cytotoxic efficiency of the drugs. The importance of timing is essential as outlined in this discovery in the context of emergence of androgen-independent, or hormonally-refractory, tumor cells. Because the population of these cells is at a minimum level at this time, and consequently easier to eliminate, or cytoreduce, the substantial reduction of any tumor cell population reduces the probability of genetic drift or evolution to even hardier tumor phenotypes. Accordingly, even if the emergent tumor type is not completely eliminated given the 60% response rate at relapse, the time to clinically detectible or symptomatic recurrence is substantially prolonged as compared to current standards of care and treatment. Thus the novel restructuring of the way patients who have advanced prostate cancer at the time of diagnosis are treated.

Although combinations of EMP and paclitaxel have been tried in various experiments, they have invariably been administered to patients in which hormone therapy has been tried and has failed.

The regimen of the subject invention is particularly novel in the requirement that tumor burden be monitored by means known in the art from the outset of hormone therapy, and that the cytotoxic chemotherapy portion of the regimen be initiated at a time of minimal tumor burden, usually 8 weeks but possibly from 2-24 weeks after the start of hormone therapy, as determined from the monitoring. Hormone therapy in the form of androgen ablation may be by surgical castration, diethylstilbestrol, anti-androgen, Ketoconizole and corticosteroids, LHRH analogs such as Lupron or Zoladex, or any combination of these or other hormonally active agents which may be developed and become available to those skilled in the art. Although examples of preferred cytotoxic combinations are provided herein, as those of skill in the art become aware of new effective cytotoxic combinations, those combinations can be substituted into the regimen of the subject invention and be administered at a time of minimal tumor burden as taught herein. The commercial embodiments of the subject invention are articles of manufacture comprising a container; a pharmaceutically acceptable cytotoxic composition disposed within the container; and, accompanying the container, instructions for administering the cytotoxic composition to a patient according to the novel scheduling and dosages disclosed herein. The instructions may either be affixed to the container or packaged with the container.

All patents and other publications cited or referred to herein are incorporated by reference as if fully set forth herein to the extent that they do not contradict the specific teachings herein. The following examples are provided by way of illustration only and not by way of limitation. Those of skill in the art will recognize a variety of non-critical parameters which could be changed or modified to yield essentially similar results without deviating from the subject invention.

### EXAMPLE 1

This strategy is based on the fact that most patients harbor a predominant androgen-dependent tumor and a limited androgen-independent tumor cell population at the time of diagnosis. Successful therapy with androgen depletion reduces the androgen-dependent population to a minimum after approximately eight weeks, although variances depending upon the degree of androgen dependence may occur, with minimal disease status ranging from one to six months. This response, if not entirely complete, sets the stage for emergence of an androgen-independent population, based on selection and/or induction, and possible regrowth of androgen-dependent tumor cells if androgen ablation therapy is stopped (12, 12, 16). Thus, the eventual relapse of most prostate cancer patients on hormonal treatment.

Non-hormonal chemotherapy is usually administered after relapse occurs, a period of increasing tumor burden and heterogeneity. Although counterintuitive, and not standard-of-care dogma, according to the subject invention the preferred time to add cytotoxic chemotherapy is when there is maximal decrease in the androgen-dependent tumor burden, minimal androgen-independent tumor burden, improved clinical performance status, and motivation on the part of the patient to accept chemotherapy. This time-treatment based strategy assumes a reasonable methodology to estimate minimal tumor burdens. Surrogate tumor markers such as prostatic specific antigen (PSA), or non-specific lactic dehydrogenase (LDH) are worthy techniques to determine their status.(11) Since smaller tumors generally respond better to cytotoxic chemotherapy than larger ones, this method extends the duration of disease control compared to most forms of sequentially administered treatments.

For purposes of the subject invention, any method which permits an objective evaluation of the level of hormone-sensitive tumor burden can be applied to determine the optimal time to begin the cytotoxic chemotherpy regimen. In cases of prostate cancer, monitoring of PSA levels is a preferred method which is well known to those of skill in the art. See, for example, Denmead et al. (1997). As a preferred example, prostate cancer patients who have begun androgen ablation therapy and present a decrease in PSA to normal levels can be viewed as having reached minimal tumor burden, and will be ready to begin cytotoxic chemotherapy according to the teachings herein. Alternatively, Kim et al. (1998) have reported serum interleukin-6 (sIL-6) levels as a predictor of early progression in androgen-independent prostate cancer (AIPC). Accordingly, by monitoring the PSA/sIL-6 ratio as taught by Kim et al., those of skill in the art can use this as an alternate marker to determine an optimal time to begin the cytotoxic chemotherapy regimen of the subject invention.

In a preferred embodiment, this invention is the use of a novel cytotoxic drug combination of paclitaxel given as a one-hour infusion at 45-120 mg/m² on days 2, 6, and 10 of a 21 -day cycle, and estramustine given orally at well-known standard daily dosages of from 280 to 840 mg/m²/d, or preferably, for example, 600 mg/m²/d, on days 1-11 of the 21-day cycle (hereinafter, the “Q4Dx3 taxol-estramustine combination”). The first cycle was initiated at minimal tumor burden, approximately 8 weeks after hormonal therapy began on patient “X”. After being treated with hormonal therapy and the Q4Dx3 taxol-estramustine combination as taught herein, patient “X” has surprisingly remained free of disease for 1-½ years. The combination was extremely well tolerated and the patient continues to receive anti-androgen therapy.

### EXAMPLE 2

Additional embodiments of the subject invention include the administration of paclitaxel over alternative schedules which are well known to those of ordinary skill in the art for the administration of paclitaxel. For example, following pre-treatment of the patients to alleviate or minimize hypersensitivity responses, paclitaxel can be infused in dosages of between about 120 mg/m² and about 275 mg/m², more preferably between about 135 mg/m² and about 175 mg/m², over a duration of about six hours or less, and preferably about one to three hours, on a 21-day cycle, beginning one day after initiation of oral administration of EMP as described in Example 1 above. Alternatively, paclitaxel dosages of between about 135 mg/m² and about 275 mg/m², preferably between about 135 mg/m² and about 175 mg/m², can be administered via a 6- to 24-hour infusion, preferably a 24-hour infusion, following premedication and on a a 21-day cycle, beginning one day after initiation of oral administration of EMP as described in Example 1 above. Such alternative modes of paclitaxel administration are taught by, for example, U.S. Pat. No. 5,621,001 issued to Canetta et al.

### EXAMPLE 3

Yet another embodiment of a combination of cytotoxic chemicals which can be used in the regimen of the subject invention is disclosed in Pienta and Smith (1997). For example, once it is observed that the initial hormone-sensitive tumor burden has been minimized, cytotoxic chemotherapy on a 21-day cycle can begin by administering estramustine at a dosage of 10 mg/kg/d orally for days 1-14 in combination with etoposide at 50 mg/m²/d orally for days 1-14, plus paclitaxel at 135 mg/m² infused over 1 hour on day 2. In a preferred variation of this embodiment, the paclitaxel is administered as a 1-hour infusion at from about 45-120 mg/m², most preferably at about 60 mg/m², on days 2, 6, and 10 of the 21-day cycle. This cycle is to be administered at least once, and is preferably repeated from 3 to 6 times, but may be repeated even more as long as cumulative toxic effects are tolerable for the patient.

### REFERENCES

(1) Panda, D. Miller, H. P, Islara, K. Wilson, L., “Stabilization of microtubule dynamics by estramustine by binding to a novel site in tubulin; a possible mechanistic basis for its antitumor action” Proc. Natl. Acad. Sci. USA Sep. 30, 1997; 94(20): 10560-10564.

(2) Peereboom, E., Klein, T., Olenki, C., Connell, R., Aaron, S. Pratt, R. Bakowski, M., “Phase I Trial of Paclitaxel and Estramustine in Hormone-Refractory Prostate Cancer,” Proc. ASCO p. 263, ab690, 1996.

(3) Hudes, G. R. , Nathan, F. E., Hater, C., Greenberg, R. Gambelia, L., Stem, C. and McAllen, C. “Paclitaxel plus estramustine in metastatic hormone refractory prostate cancer.” Seminars in Oncology, Vol 22, 41-45, 1995

(4) Gray, C. Roth, B. Hudes, G. Babb, J. et al. “Phase I trial of weekly paclitaxel by 3-Hour infusion plus oral estramustine in metastatic hormone-refractory cancer.”Proc. ASCO, Pg. 331a, ab 1278, 1998.

(5) Perry, C. M., McTavish, D. “Estamustine phosphate sodium. A review of its pharmacodynamic and pharmaokinetic properties, and therapeutic efficacy in prostate cancer.” Drugs Aging Jul. 7, 1995:1 49-74

(6) Hartley-Asp, B. Krose, E. “Nuclear protein matrix as a target for estramostine-induced cell death.” Prostate 1986 9:4 387-95.

(7) Vallbo, C. Bergenhelm, T. Bergh, A. Grankvist, K. Henriksson, R. “DNA fragmentation induced by the antimitotic drug estramustine in malignant rat glioma but not In normal brain—suggesting an apoptotic cell-death.” Br. J. Cancer April 1995 71:4 717-20

(8) Dahllof, G. Billstrom, A. Cabral, F., Hatley-Asp B. “Estramustine depolymerizes microtubules by binding to tubulin.” Cancer Res. Oct. 1, 1993 53:19 4573-81.

(9) Speicher, L. A., Barone, L. Tew, K. D., “Combined antimicrotubule activity of estramustine and taxol in human prostatic carcinoma cell lines.” Cancer Res. Aug. 15, 1992 52:16 4433-40.

(10) Hudes, G. R., Nathan, F., Chater, C. Haas, N. Cornfield, M. Giantonio, B. Greenberg, R., Gomella, L. Litwin, S., Ross, E., Roethke, S., McAleer, C. “Phase II trial of 96-hour paclitaxel plus oral estramustine phosphate in metastatic honnone-refractory prostate cancer.” J. Clin Oncol. September 1997 15:9 3156-63.

(11) Denmead, S. R., Lou, W., Malm, J. et al. “Specific and efficient peptide substrates for assaying the proteolytic activity of prostate specific antigen.” Cancer Res. 57:4920-4926, 1997.

(12) Koivisto, P., Kolmer, M. Visakorpi, T., Kallionlemi, O.P. “Androgen receptor gene and hormonal therapy failure of prostate cancer.” Am J. Path, 152:1-9, 1998.

(13) Perry, J. E., and Tindall, D. J., “Androgens regulate the expression of proliferating cell nuclear antigen post-transcriptionally in the human prostate cancer cell line LnoaP.” Cancer Research 57:1539-1544.:

(14) Helson, L., Helson, C., Puccio, M., Ainsworth, S. Pilla, P. “Designing drug dosing according to cytotoxic effects of taxol in neuroblastoma cell lines.” Stonybrook Symposium on Taxol, May 14-15, Stonybrook, N.Y., 1993.

(15) Helson, L, “Clinical results of a Q4 dayx 3 Taxol regimen.” Cancer Investigation 12:30-31, 1993.

(16) Ainsworth et al., U.S. Pat. No. 5,696,153

(17) Chen, C. T., Gan, Y., Au, J. L. S., Wientjes, M. G “Androgen-dependent and independent human prostate xenograft tumors as models for drug activity evaluation.” Cancer Research 58:2777-2783, 1998

(18) J. Kim, L. Finn, S. Hodges, N. Hooseln, L. Deltos, C. Logothetis, “Neuroendocrine Marker as a Predictor of Early Progression in Androgen-Independent Prostate Cancer” (AIPC) Proceedings of ASCO Vol. 17, 1998 ab 1213

1. A method of treating a patient having symptoms ofprostate cancer, comprising the steps of:
initiating androgen ablation;
monitoring the patient's tumor burden; and
initiating chemotherapy with at least one cytotoxic composition at a time of minimal tumor burden to the patient.

2. A method according to claim 1, wherein said monitoring step comprises monitoring of at least one compound selected from the group consisting of prostatic specific antigen, lactic dehydrogenase, and serum interleukin-6.

3. A method according to claim 1, wherein said initiating chemotherapy step is at a time of from 2 weeks to 6 months after initiating androgen ablation.

4. A method according to claim 3, wherein said initiating chemotherapy step is at a time of about 8 weeks after initiating androgen ablation.

5. A method according to claim 1, wherein said cytotoxic composition comprises a taxane.

6. A method according to claim 2, wherein said cytotoxic composition comprises a taxane.

7. A method according to claim 3, wherein said cytotoxic composition comprises a taxane.

8. A method according to claim 4, wherein said cytotoxic composition comprises a taxane.

9. A method according to claim 5, wherein said taxane is paclitaxel.

10. A method according to claim 6, wherein said taxane is paclitaxel.

11. A method according to claim 7, wherein said taxane is paclitaxel.

12. A method according to claim 8, wherein said taxane is paclitaxel.

13. A method according to claim 1, wherein said cytotoxic composition comprises estramustine.

14. A method according to claim 2, wherein said cytotoxic composition comprises estramustine.

15. A method according to claim 3, wherein said cytotoxic composition comprises estramustine.

16. A method according to claim 4, wherein said cytotoxic composition comprises estramustine.

17. A method according to claim 1, wherein said cytotoxic composition comprises etoposide.

18. A method according to claim 2, wherein said cytotoxic composition comprises etoposide.

19. A method according to claim 3, wherein said cytotoxic composition comprises etoposide.

20. A method according to claim 4, wherein said cytotoxic composition comprises etoposide.

21. A method according to claim 9, wherein said chemotherapy comprises the administration of paclitaxel at a dosage of from 45-135 mg/m² over a duration of from 60-180 minutes for a plurality of times during a 21-day period.

22. A method according to claim 10, wherein said chemotherapy comprises the administration of paclitaxel at a dosage of from 45-135 mg/m² over a duration of from 60-180 minutes for a plurality of times during a 21-day period.

23. A method according to claim 11, wherein said chemotherapy comprises the administration of paclitaxel at a dosage of from 45-135 mg/m² over a duration of from 60-180 minutes for a plurality of times during a 21-day period.

24. A method according to claim 12, wherein said chemotherapy comprises the administration of paclitaxel at a dosage of from 45-135 mg/m² over a duration of from 60-180 minutes for a plurality of times during a 21-day period.

25. A method acccording to claim 21, wherein each of said 60-180 minute administrations is separated from the next by an interval of from about 4 to 5 days.

26. A method according to claim 22, wherein each of said 60-180 minute administrations is separated from the next by an interval of from about 4 to 5 days.

27. A method according to claim 23, wherein each of said 60-180 minute administrations is separated from the next by an interval of from about 4 to 5 days.

28. A method according to claim 24, wherein each of said 60-180 minute administrations is separated from the next by an interval of from about 4 to 5 days.

29. A method according to claim 25, wherein the dosage of paclitaxel is about 60 mg/m² and the administrations are on days 2, 6, and 10 of a 21-day cycle.

30. A method according to claim 9, wherein said chemotherapy comprises the administration of paclitaxel at a dosage of from about 120 mg/m² to about 275 mg/m² for a duration of about 6 hours or less.

31. A method according to claim 10, wherein said chemotherapy comprises the administration of paclitaxel at a dosage of from about 120 mg/m² to about 275 mg/m² over a duration of about 6 hours or less.

32. A method according to claim 11, wherein said chemotherapy comprises administration of paclitaxel at a dosage of from about 120 mg/m² to about 275 mg/m² over a duration of about 6 hours or less.

33. A method according to claim 12, wherein said chemotherapy comprises the administration of paclitaxel at a dosage of from about 120 mg/m² to about 275 mg/m² over a duration of about 6 hours or less.

34. A method according to claim 13, wherein said dosage of paclitaxel is from about 135 mg/m² to about 175 mg/m².

35. A method according to claim 31, wherein said dosage of paclitaxel is from about 135 mg/m² to about 175 mg/m².

36. A method according to claim 32, wherein said dosage of paclitaxel is from about 135 mg/m² to about 175 mg/m².

37. A method according to claim 33, wherein said dosage of paclitaxel is from about 135 mg/m² to about 175 mg/m².

38. A method according to claim 9, wherein said chemotherapy comprises the administration of paclitaxel at a dosage of from about 120 mg/m² to about 275 mg/m² over a duration of about 6-24 hours.

39. A method according to claim 10, wherein said chemotherapy comprises the administration of paclitaxel at a dosage of from about 120 mg/m² to about 275 mg/m² over a duration of about 6-24 hours.

40. A method according to claim 11, wherein said chemotherapy comprises the administration of paclitaxel at a dosage of from about 120 mg/m² to about 275 mg/m² over a duration of about 6-24 hours.

41. A method according to claim 12, wherein said chemotherapy comprises the administration of paclitaxel at a dosage of from about 120 mg/m² to about 275 mg/m² over a duration of about 6-24 hours.

42. A method according to claim 38, wherein said dosage of paclitaxel is from about 135 mg/m² to about 175 mg/m².

43. A method according to claim 39, wherein said dosage of paclitaxel is from about 135 mg/m² to about 175 mg/m².

44. A method according to claim 40, wherein said dosage of paclitaxel is from about 135 mg/m² to about 175 mg/m².

45. A method according to claim 41, wherein said dosage of paclitaxel is from about 135 mg/m² to about 175 mg/m².

46. A method according to claim 13, wherein said chemotherapy comprises the administration of estramustine at a dosage of from 280-840 mg/m²/d on days 1-11 of a 21-day cycle.

47. A method according to claim 14, wherein said chemotherapy comprises the administration of estramustine at a dosage of from 280-840 mg/m²/d on days 1-11 of a 21-day cycle.

48. A method according to claim 15, wherein said chemotherapy comprises the administration of estramustine at a dosage of from 280-840 mg/m²/d on days 1-11 of a 21-day cycle.

49. A method according to claim 16, wherein said chemotherapy comprises the administration of estramustine at a dosage of from 280-840 mg/m²/d on days 1-11 of a 21-day cycle.

50. A method according to claim 46, wherein the dosage of estramustine is about 600 mg/m²/d.

51. A method according to claim 47, wherein the dosage of estramustine is about 600 mglm²/d.

52. A method according to claim 48, wherein the dosage of estramustine is about 600 mg/m²/d.

53. An method according to claim 49, wherein the dosage of estramustine is about 600 mg/m²/d.

54. A method according to claim 17, wherein said chemotherapy comprises the administration of etoposide at a dosage of about 50 mg/m²/d on days 1-14.

55. A method according to claim 18, wherein said chemotherapy comprises the administration of etoposide at a dosage of about 50 mg/m²/d on days 1-14.

56. A method according to claim 19, wherein said chemotherapy comprises the administration of etoposide at a dosage of about 50 mg/m²/d on days 1-14.

57. A method according to claim 20, wherein said chemotherapy comprises the administration of etoposide at a dosage of about 50 mg/m²/d on days 1-14.

58. A method according to claim 21, wherein said chemotherapy comprises the administration of paclitaxel according to the Q4Dx3 taxol-estramustine combination.

59. A method according to claim 22, wherein said chemotherapy comprises the administration of paclitaxel according to the Q4Dx3 taxol-estramustine combination.

60. A method according to claim 23, wherein said chemotherapy comprises the administration of paclitaxel according to the Q4Dx3 taxol-estramustine combination.

61. A method according to claim 24, wherein said chemotherapy comprises the administration of paclitaxel according to the Q4Dx3 taxol-estramustine combination.
